(19) United States
(12) Patent Application Publication
(10) Pub. No.: US 2011/0151395 A1
(43) Pub. Date: Jun. 23, 2011

(54) LIGHT EMITTING DEVICE AND DENTAL HANDPIECE DEVICE

(75) Inventors: Kei Ishihara, Kyoto (JP); Makoto Numakawa, Kyoto (JP); Hirofumi Jikuhara, Kyoto (JP)

(73) Assignee: J. MORITA MANUFACTURING CORPORATION, Kyoto-Shi (JP)

(21) Appl. No.: 12/976,083

(22) Filed: Dec. 22, 2010

(30) Foreign Application Priority Data
Dec. 23, 2009 (JP) 2009-291579

(51) Int. Cl. A61B 1/24

(52) U.S. Cl. 433/29

(57) ABSTRACT

A light emitting device is provided in a dental handpiece device for illuminating an area in the vicinity of a dental care tool at a tip of the dental handpiece device. The light emitting device includes a light emitting section including a light emitting diode element and an electrode, and an electric power control section for controlling electric power to be supplied to the light emitting diode element via the electrode. The electric power control section includes an electric current restriction element for restricting an amount of electric current to be supplied to the light emitting diode element, and a reverse voltage prevention element for preventing a reverse voltage from being applied to the light emitting diode element.

### BACKGROUND OF THE INVENTION

1. Field of the Invention

The present invention relates to a dental handpiece device usable for medical care in the state where, for example, a dental care tool is attached to a tip of the dental handpiece device and acts on a tooth; and also to a light emitting device, a light emitting diode illumination component and a connection component usable for such a dental handpiece device.

2. Description of the Prior Art

A dental handpiece device is used for medical care in the state where a dental care tool (cutting tool, scaler or the like) is attached to a tip head section and acts on a tooth. Therefore, the dental handpiece is structured such that a driving medium for actuating the tool (air, water, electric power or the like) is supplied to a driving body of the tool (air turbine, micromotor, ultrasonic oscillator or the like) via a handpiece main body of the dental handpiece device.

In more detail, the dental handpiece device includes a flexible tube having a built-in driving medium pathway, a connection component connectable to the flexible tube, and a handpiece main body detachably connectable to a tip of the connection component.

In the handpiece main body, a driving medium pathway is formed for supplying the driving medium for actuating the dental care tool. In the connection component, a driving medium communication pathway is formed for communicating these two driving medium pathways when the flexible tube and the handpiece main body are connected to each other. A connection part of the handpiece main body connectable to the connection component is structured so that the handpiece main body connected to the connection component is pivotable around an axis and so the handpiece main body, which is to be held by the operator, is easy to handle.

Such a dental handpiece device is used for a precision work of treating a tooth in a dark and small area of the oral cavity. Therefore, illumination is necessary for the site to be treated.

In this situation, a dental handpiece device with a connection component, in which an LED for illuminating the target site to be examined is provided in a neck part of a handpiece head, is disclosed (see Patent Document 1). In this dental handpiece device, the LED is provided at a position in the handpiece main body and in the vicinity of a dental care tool, and the LED illuminates the target site of the medical care. According to the disclosed structure, an electric cord from the LED is electrically connected to an electric cord of the power source at the connection part, of the handpiece main body, connected with the connection component (coupling).

A dental treating tool including an illumination mechanism which has a multicolor LED having green, red and blue LED components as a light source is also disclosed (see Patent Document 2). This dental treating tool includes a voltage regulation circuit and is described as being capable of changing the color tone of the light emitted by the multicolor LED in accordance with the type of the treatment. It is also disclosed that in a connection component (coupling) in this dental treating tool, a conventional halogen lamp is also usable instead of the multicolor LED.

An LED lamp for restricting the amount of electric current to be supplied to the LED and thus preventing heat generation in the LED, using an electric current restriction element provided in the electric circuit is also disclosed (see Patent Document 3).

However, the dental handpiece device described in Patent Document 1 is usable only with a handpiece having a built-in LED. Therefore, there is a problem that an existing light-guide-type handpiece is not usable.

The dental treating tool described in Patent Document 2 uses the voltage regulation circuit and so has a problem that the luminosity of the LED lamp varies. In more detail, in the case where the voltage regulation circuit is used, the electric current level is always the same when the LED lamp is turned on from an off state because the voltage level is the same. However, as the time passes, the electric current level changes due to heat generation caused in the LED. As a result of such a change of the electric current level, the luminosity varies.

There is another problem that the level of heat generation varies in accordance with the color of the light emitted by the LED, and so the color tone of the light gradually changes while the tool is being used.

There is still another problem that the shape of the terminal of the LED lamp is different from that of the conventional halogen lamp, and so the LED lamp is not usable in an existing device.

The LED lamp described in Patent Document 3 is conceived for the purpose of suppressing a change of the wavelength of the emitted light caused by heat generation, using an electric current regulation device provided in the electric circuit.

Such a change of the wavelength is a problem occurring when a high luminance LED chip and a general-purpose LED chip are both used. The electric current regulation device in the electric circuit described in this publication is not provided for the purpose of maintaining the luminosity at a certain level.

There is still another problem that when an LED lamp is inserted into a socket in a reverse, wrong direction, the LED lamp receives an excessive voltage and is broken.

### PRIOR ART DOCUMENT

### Patent Document

Patent Document 1: Japanese Laid-Open Patent Publication No. 2008-307248

Patent Document 2: Japanese Laid-Open Patent Publication No. Hei 7-328032

Patent Document 3: Japanese Laid-Open Patent Publication No. 2003-273404

### SUMMARY OF THE INVENTION

### Problems to be Solved by the Invention

The present invention made in light of the above-described problems has an object of providing a light emitting device, a light emitting diode illumination component, a connection component and a dental handpiece device which prevent the luminosity from varying even when adopted for a dental unit usable at any of various voltage levels and also prevent an LED from being broken as a result of the LED being inserted in the wrong direction.

### Means for Solving the Invention

The present invention is directed to a light emitting device provided in a dental handpiece device for illuminating an area in the vicinity of a dental care tool at a tip of the dental handpiece device, the light emitting device comprising a light emitting section including a light emitting diode element and an electrode; and an electric power control section for controlling electric power to be supplied to the light emitting diode element via the electrode. The electric power control section includes an electric current restriction element for restricting an amount of electric current to be supplied to the light emitting diode element; and a reverse voltage prevention element for preventing a reverse voltage from being applied to the light emitting diode element.

Owing to this, the electric current to be supplied to the light emitting diode element can be maintained at a certain level by the electric current restriction element, and whichever level of voltage may be applied among various voltage levels, the color tone and the luminosity of the light from the light emitting diode element can be maintained at a certain level. The reverse voltage prevention element can protect the light emitting diode element against a reverse voltage.

Even when the light emitting diode element is connected in a wrong direction in terms of the positive and negative poles, the light emitting diode element can be prevented from being broken.

The present invention is also directed to a light emitting diode illumination component acting as the light emitting device, in which the light emitting section and the electric power control section are integrated with each other; and a socket connection section connectable to a socket in the dental handpiece device is provided.

Owing to this, the light emitting diode illumination component can be used as being attached to the socket in a conventional dental handpiece device. Namely, the light emitting diode illumination component according to the present invention can protect the light emitting diode element against various environments; for example, where the light emitting diode element is usable at any of various voltage levels or is connected in the wrong direction in terms of the positive and negative poles. Therefore, the light emitting device according to the present invention can be attached to, and used in, a conventional dental handpiece device which generally uses a halogen lamp as the light emitting device.

When a halogen lamp is used, it does not matter in whichever direction the lamp may be connected in terms of the positive and negative poles. Therefore, the halogen lamp is usually connectable in either way. The light emitting device using the light emitting diode element according to the present invention also causes no problem even if being connected in the wrong direction, owing to the above-described structure. The light emitting diode element does not emit light but is not broken either, and can be re-attached in the correct direction and used with no problem.

Even if an appropriate amount of electric current is not supplied, the electric current regulation element can adjust the amount to be appropriate. Therefore, the color tone or the luminosity of the light from the light emitting diode element does not reach an unexpected level, and the light emitting diode element can be used properly.

In an embodiment of the present invention, a substrate for carrying the electric power control section thereon is provided, and the light emitting diode element, at least a part of the electric power control section, the substrate, and the socket connection section may be physically arranged in series in this order and fixed.

The at least apart of the electric power control section preferably includes a relatively large element, such as an electric current regulation element, among elements provided as the electric power control section.

Owing to this embodiment, the light emitting diode illumination component can be structured to be compact, and so can be mounted in a small space of the dental handpiece device in which the halogen lamp is conventionally located.

The present invention is also directed to a connection component comprising a light emitting device and having a handpiece main body connected to one end thereof, and thus being included in a dental handpiece device, the connection component comprising a tip-side connection section provided at the one end for allowing the handpiece main body to be connected thereto detachably and pivotably around an axis; a rear-side connection section provided at another end of the connection component for allowing a tube to be connected thereto; a driving medium pathway provided inside the connection component for allowing a driving medium supplied from the tube via the rear-side connection section to pass therethrough and reach the handpiece main body; and a socket provided at a tip of the tip-side connection section for allowing the light emitting section to be attached thereto in the state where the light emitting section faces a rear end of a waveguide body provided in the handpiece main body.

The driving medium may be an appropriate medium for driving a driving body provided in a head section or the like of the handpiece main body, for example, air, water, electric power or the like.

Owing to this, a connection component using a light emitting diode element as the light emitting device can be provided.

In an embodiment of the present invention, the electric power control section may be provided rearward with respect to the tip-side connection section.

The power control section may include a rectifier circuit and a constant electric current circuit or include a constant electric current circuit and a reverse voltage circuit.

Owing to this embodiment, the mounting space of the electric power control section can be made larger than that in the case where the electric power control section is integrated in the light emitting diode illumination component. Namely, in the case where the electric power control section is included in the light emitting diode illumination component, the electric power control section is inevitably mounted in an area facing a waveguide body of the handpiece main body. Therefore, the mounting space is very small.

The tip-side connection section of the connection component does not have an extra space almost at all because the pathways for the electric cords provided for supplying the electric power to the light emitting diode element and the pathways for the driving mediums gather in this section. Therefore, mountable devices are limited, and it is difficult to mount a device of a certain size such as the rectifier circuit. By contrast, in the case where the electric power control section is provided at a position rearward with respect to the tip-side connection section, a wide mounting space for the electric power control section is obtained and so a relatively large device such as the rectifier circuit is made mountable.

The present invention is also directed to a dental handpiece device comprising the connection component and a handpiece main body connectable to the tip-side connection section of the connection component detachably and pivotably around an axis. The handpiece main body includes a tip head section provided at a tip thereof for allowing a dental care tool to be attached thereto; a driving medium pathway provided inside the handpiece main body for allowing a driving medium, supplied for driving a dental care tool, to pass therethrough; and a waveguide body for guiding light from the light emitting diode element of the connection component and directing the light from an area in the vicinity of the tip head section toward an area in the vicinity of the dental care tool.

Owing to this, a dental handpiece device using a light emitting diode element as the light emitting device can be provided.

### Effect of the Invention

The present invention can prevent the luminosity from varying at whichever voltage level a dental unit may be used among various voltage levels, and also prevent an LED from being broken as a result of the LED being inserted in a wrong direction. The present invention also allows an operator to use the illumination by a low-cost and durable LED even with an existing handpiece.

### BRIEF DESCRIPTION OF DRAWINGS

FIG. 1 is a side view showing a schematic structure of a dental handpiece device in Example 1;

FIG. 2 is a partially exploded vertical cross-sectional view of a connection component of the dental handpiece device;

FIG. 3A is a front view of a light emitting diode illumination component;

FIG. 3B is a bottom view of the light emitting diode illumination component;

FIG. 3C is a cross-sectional view taken along line A-A in FIG. 3B as seen in the direction of the arrows;

FIG. 3D is a cross-sectional view taken along line B-B in FIG. 3C as seen in the direction of the arrows;

FIG. 3E is a cross-sectional view taken along line C-C in FIG. 3C as seen in the direction of the arrows;

FIG. 3F is a cross-sectional view taken along line D-D in FIG. 3C as seen in the direction of the arrows;

FIG. 4 is a block diagram showing a circuit configuration of the light emitting diode illumination component;

FIG. 5 is a partially cross-sectioned side view of a dental handpiece device;

FIG. 6A is a front view of a connection component in Example 2;

FIG. 6B is a bottom view of the connection component in Example 2;

FIG. 7 is a partially exploded cross-sectional view of the connection component in Example 2 taken along line E-E in FIG. 6B as seen in the direction of the arrows;

FIG. 8 is a cross-sectional view taken along line F-F in FIG. 6B as seen in the direction of the arrows; and

FIG. 9 is a circuit block diagram of an electric power control section in Example 2.

### DESCRIPTION OF THE PREFERRED EMBODIMENTS

One embodiment of the present invention will be described with reference to the drawings.

### EXAMPLE 1

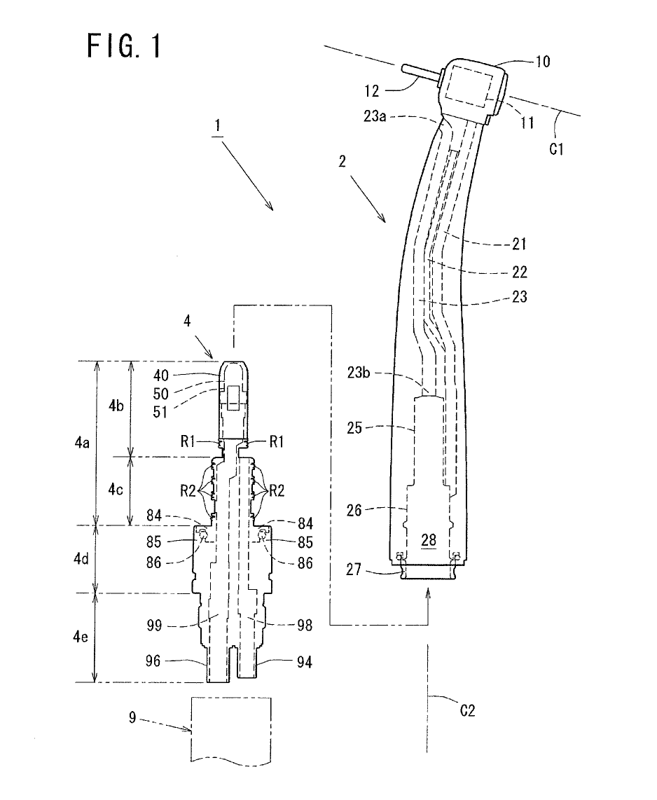

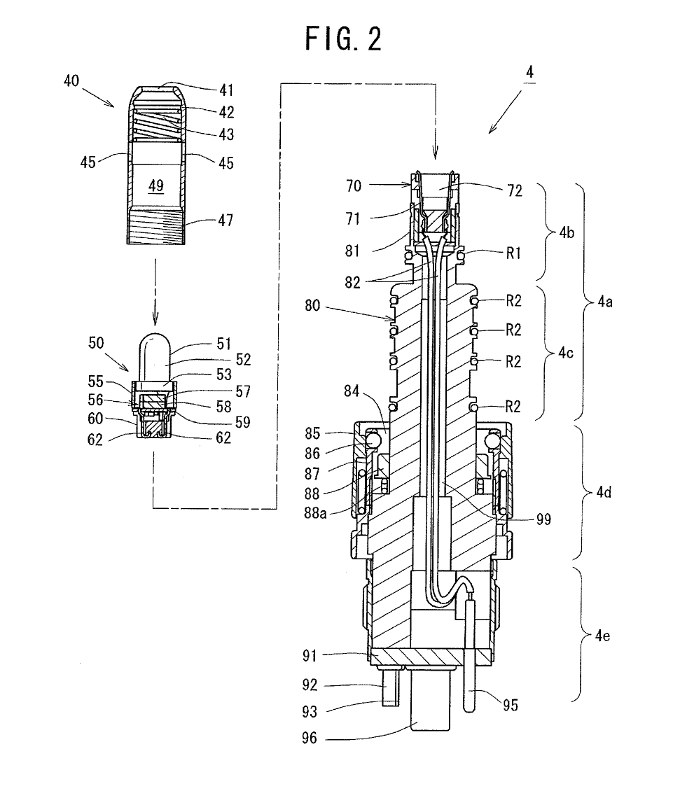

FIG. 1 is a side view showing a schematic structure of a dental handpiece device 1 in Example 1. FIG. 2 is a partially exploded vertical cross-sectional view of a connection component 4 of the dental handpiece device 1.

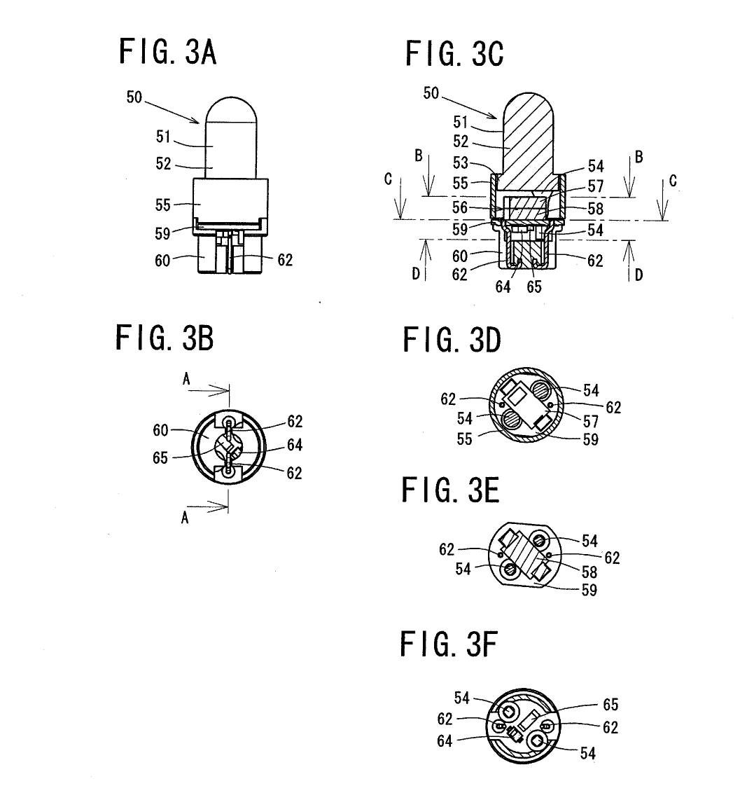

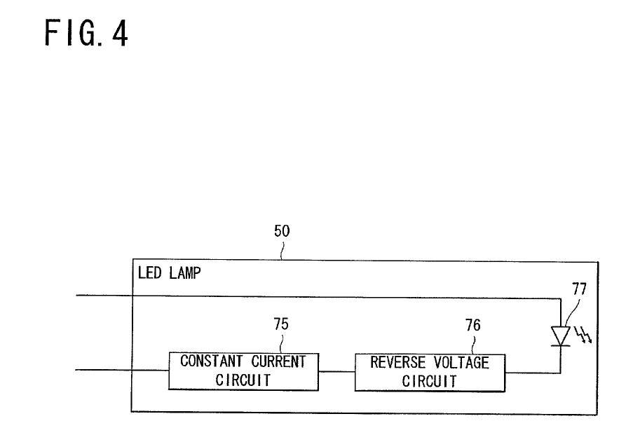

FIG. 3A is a front view of the light emitting diode illumination component 50, and FIG. 3B is a bottom view thereof. FIG. 3C is a cross-sectional view taken along line A-A in FIG. 3B as seen in the direction of the arrows. FIG. 3D is a cross-sectional view taken along line B-B in FIG. 3C as seen in the direction of the arrows. FIG. 3E is a cross-sectional view taken along line C-C in FIG. 3C as seen in the direction of the arrows. FIG. 3F is a cross-sectional view taken along line D-D in FIG. 3C as seen in the direction of the arrows. FIG. 4 is a block diagram showing a circuit configuration of the light emitting diode illumination component 50.

As shown in FIG. 1, the dental handpiece device 1 mainly includes a handpiece main body 2 and the connection component 4.

The handpiece main body 2 is formed to have a generally cylindrical shape and gradually tapers off from a base part (bottom in the figure) toward a tip part (top in the figure) while slightly warping toward a rear side thereof (right in the figure). The handpiece main body 2 includes a head section 10 at a tip thereof (top end in the figure).

Inside the head section 10, a driving body 11 is provided. The driving body 11 includes an air turbine, a micromotor, an ultrasonic oscillator or the like, namely, driving means using an appropriate driving medium such as air, water, electric power or the like. In the case where the driving body 11 is an air turbine, when air is supplied as the driving medium, the air turbine is driven to rotate by a pressure of the air. In the case where the driving body 11 is a micromotor, when an electric power is supplied, the micromotor is driven to rotate. The driving body 11 may be provided in a grasping part of the handpiece main body 2, instead of inside the head section 10.

To the driving body 11, a dental care tool 12 is attached along a rotation axis C1. The dental care tool 12 projects to the outside of the head section 10. The dental care tool 12 may be any appropriate tool usable for medical care (diagnosis or treatment) such as a cutting tool, a scaler or the like. In the case where the dental care tool 12 is a cutting tool, an air turbine or a micromotor is preferably usable as the driving body 11. In the case where the dental care tool 12 is a scaler, an ultrasonic oscillator is preferably usable as the driving body 11.

The handpiece main body 2 includes a connection ring 27 in the base part thereof. The connection ring 27 is inserted into an attachment space 84 of the connection component 4 and is rotatably attached to the connection component 4 in the state where an outer concaved surface of the connection ring 27 is in contact with a ball 86 of the connection component 4.

Inside the base part of the handpiece main body 2, a large diameter cylindrical wall 26 and a small diameter cylindrical wall 25 are located in this order from the connection ring 27 toward the tip part (top in the figure). An inner space of the small diameter cylindrical wall 25 and the large diameter cylindrical wall 26 acts as an attachment hole 28. Into the attachment hole 28, a tip-side connection section 4a of the connection section 4 is inserted. The tip-side connection section 4a includes a small diameter section 4b, which is inserted into the inner space of the small diameter wall 25 and is sealed with an O-ring R1 (see FIG. 2). The tip-side connection section 4a also includes a large diameter section 4c, which is inserted into the inner space of the large diameter wall 26 and is sealed with an O-ring R2 (see FIG. 2).

Inside the handpiece main body 2, a driving medium pathway 21, a working medium pathway 22, and a waveguide body 23 are located from the tip part to a position which is slightly closer to the base part with respect to the center of the handpiece main body 2.

The driving medium pathway 21 is a pathway for transporting a driving medium such as air, electric power or the like supplied from the tube 9 via the connection component 4. The transported driving medium is supplied to the driving body 11.

The working medium pathway 22 is a pathway for transporting a working medium such as cleaning water, cooling air or the like supplied from the tube 9 via the connection component 4. The transported working medium is ejected toward a target site of the medical care at a tip of the dental care tool 12 in the head section 10.

The waveguide body 23 is formed of an optical fiber, and a base-side end 23b thereof is located to face a tip of alight emitting diode section 51 of the connection component 4 attached to the handpiece main body 2. The base-side end 23b is formed to be straight along a rotation axis C2 around which the handpiece main body 2 and the connection component 4 are rotatable. Owing to such a shape, the light emitted by the light emitting diode section 51 can be efficiently guided.

A tip-side end 23a of the waveguide body 23 is located in the vicinity of the dental care tool 12 in the head section 10. The tip-side end 23a is directed to a position in the vicinity of the tip of the dental care tool 12. Therefore, the light which has passed through the waveguide body 23 is directed from the tip-side end 23a toward a position in the vicinity of the tip of the dental care tool 12 obliquely with respect to the rotation axis.

As shown in FIG. 2, the connection component 4 includes a connection component main body 80, the light emitting diode illumination component 50 attached to a tip thereof, and a cover 40 for covering the light emitting diode illumination component 50.

The cover 40 is formed to have a generally cylindrical shape and is capable of accommodating the light emitting diode illumination component 50. A tip part of the cover 40 is internally inclined, and an illumination hole 41 is formed at a center thereof. Through the illumination hole 41, the light from the light emitting diode section 51 is directed.

Inside of the tip part of the cover 40, a compression coil spring 43 is provided. The compression coil spring 43 is located along an inner circumferential surface of the cover 40, and a tip thereof is stopped by a projection 42 provided inside the tip part of the cover 40. The compression coil spring 43 has such a size that allows a cannonball-shaped section 52 of the light emitting diode section 51 to pass therethrough but does allow a flange section 53 of the light emitting diode section 51 to pass therethrough. Owing to this, the compression coil spring 43 can urge the light emitting diode illumination component 50 and alleviates an impact or the like to prevent the light emitting diode illumination component 50 from being broken.

In a side surface of the cover 40, a ventilation hole 45 is formed for heat radiation.

In an inner surface of a base part of the cover 40, a female thread 47 is formed. Owing to the female thread 47, the cover 40 is engaged with a male thread formed on an outer surface of a socket attachment section 81 of the connection component main body 80.

The cover 40 maybe coated with an insulating material such as fluorine or the like in order to be insulating. Alternatively, the cover 40 itself may be molded of an insulating material such as a resin or the like.

The light emitting diode illumination component 50 mainly includes the light emitting diode section 51, a substrate 59, and an insertion section 60. The light emitting diode section 51, constant electric current diodes 57 and 58, the substrate 59, and connection terminals 62 are provided in this order. The constant electric current diodes 57 and 58 act as electric current restriction elements. The constant electric current diodes 57 and 58, a diode 64, and a resistor 65 are included in an electric power control section 56.

The connection component main body 80 mainly includes the tip-side connection section 4a, a middle section 4d, and a rear-side connection section 4e.

The tip-side connection section 4a includes the large diameter section 4c in a base part and the small diameter section 4b in a tip part.

At a tip of the small diameter section 4b, the socket attachment section 81 is provided. The socket attachment section 81 is generally cylindrical and has the male thread on an outer circumferential surface thereof. At a position slightly closer to abase section of the small diameter section 4b with respect to the socket attachment section 81, the O-ring R1 for sealing is provided.

Inside the socket attachment section 81, a socket 70 is attached. The socket 70 has an attachment hole 72 at a tip thereof. The attachment hole 72 is usable for attaching the light emitting diode illumination component 50. The socket 70 may have such a shape that allows a conventional halogen lamp to be attached thereto.

On an inner surface of the socket attachment section 81 enclosing the attachment hole 72, two connection terminals 71 are located to face each other. The connection terminals 71 respectively contact and are electrically connected to the two connection terminals 62 of the light emitting diode illumination component 50 attached to the connection component 4.

Inside the connection component main body 80, an electric cord pathway 99 is formed from the tip to a base thereof for allowing electric cords 82 to pass therethrough. In the electric cord pathway 99, two electric cords, one positive and one negative, are located. Also inside the connection component main body 80, a driving medium pathway 98 (see FIG. 1) and a working driving pathway (not shown) are formed.

The driving medium pathway 98 is communicated to the driving medium pathway 21 of the handpiece main body 2, and supplies a driving medium such as air, water, electric power or the like fed from the tube 9 to the head section 10 of the handpiece main body 2.

The working medium pathway (not shown) is communicated to the working medium pathway 22 of the handpiece main body 2, and supplies a medium such as cooling air, cleaning water or the like fed from the tube 9 to the head section 10 of the handpiece main body 2.

The large diameter section 4c is of a cylindrical shape having a larger radius than the small diameter section 4b, and has a plurality of O-rings R2 around an outer circumferential surface thereof. Inside the large diameter section 4c, an electric cord pathway 99 for allowing the electric cords 82 to pass therethrough and the like are provided.

The middle section 4d has a stepped part at which the radius of the cylindrical shape continued from the large diameter section 4c is increased in the middle. At a position on the side of the large diameter section 4c with respect to the stepped part, a cylindrical contact cushioning ring 88 is provided via urging means 88a.

Outer to the cylindrical contact cushioning ring 88, a generally cylindrical ball holding ring 87, and the ball 86 is held in a ball holding hole of the ball holding ring 87.

Outer to the ball holding ring 87, a generally cylindrical operation sleeve 85 is rotatably provided.

The middle section 4d having such a structure has the attachment space 84 between the ball 86 held by the ball holding ring 87 and the connection component main body 80. The connection ring 27 of the handpiece main body 2 is inserted into the attachment space 84, and thus the handpiece main body 2 is rotatably attached to the connection component 4. When the inserted connection ring 27 contacts the contact cushioning ring 88, an impact is applied on an end of the inserted connection ring 27, but the impact is alleviated by an urging force of the urging means 88a.

The rear-side connection section 4e has a bottom plate 91 provided at a rear end thereof. A cooling air supply insertion plug 92, a water supply insertion plug 93, an air supply insertion plug 94, an electric power plug terminal 95, and an air discharge insertion plug 96 are provided so as to project from the bottom plate 91.

As described above, the light emitting diode illumination component 50 mainly includes the light emitting diode section 51, the substrate 59, and the insertion section 60.

As shown in FIG. 3C, the light emitting diode section includes the cannonball-shaped section 52 having a spherical surface at an end of the cylindrical shape and the flange section 53 provided at the other end of the cannonball-shaped section 52 and having a large radius.

An outer circumferential surface of the flange section 53 of the light emitting diode section 51 is covered with a cylindrical covering member 55. The covering member 55 has a greater height than that of the flange section 53, and a top part of the covering member 55 faces and covers the flange section 53. A rear end of the covering member 55 is covered with the substrate 59. Inside the covering member 55, the constant electric current diodes 57 and 58 are provided on a top surface of the substrate 59. Also inside the covering member 55, connection terminals 54 as legs of the light emitting diode section 51 are provided.

The substrate 59 has an oblong shape obtained by cutting off two arcked parts of a circle in parallel.

On a bottom surface of the substrate 59, the diode 64 and the resistor 65 are provided. The diode 64 acts as a reverse voltage circuit. The two connection terminals 62 are projected from the bottom surface of the substrate 59. These two connection terminals 62 are located symmetrical with respect to the insertion section 60.

The insertion section 60 is formed of a resin, and the connection terminals 62 are located on a side surface thereof. The insertion section 60 has a convexed shape so as to be attachable to the socket 70 and preferably has the same size as that of a conventional halogen lamp which can be attached to the socket 70.

The light emitting diode illumination component 50 has a structure in which the constant electric current diodes 57 and 58 are enclosed by the flange section 53, the substrate 59, and the covering member 59 of the light emitting diode section 51. The enclosed inner space is filled with a molded resin.

FIG. 4 is a block diagram showing a structure of the light emitting diode illumination component 50.

In the light emitting diode illumination component 50, a constant electric current circuit 75, a reverse voltage circuit 76, and a light emitting diode element 77 are connected.

The constant electric current circuit 75 includes the constant electric current diodes 57 and 58 (see FIG. 3), and stabilizes the electric current to be supplied to the light emitting diode element 77 at a certain level. Owing to this, even if the voltage level varies depending on the dental unit for which the light emitting diode illumination component 50 is used, the electric current to be supplied to the light emitting diode element 77 can be kept at a certain level, and thus the color and the luminosity of the light emitted by the light emitting diode element 77 can be kept at a certain level.

The reverse voltage circuit 76 includes the diode 64 (see FIG. 3). Owing to the reverse voltage circuit 76, even when the light emitting diode illumination component 50 is attached to the socket 70 a wrong direction in terms of the positive and negative poles, the light emitting diode element 77 is prevented from being broken by a reverse electric current. When being attached to the socket 70 in the wrong direction in terms of the positive and negative poles, the light emitting diode illumination component 50 does not emit light. Therefore, the user can re-attach the light emitting diode illumination component 50 to the socket 70 in the correct direction and thus use the light emitting diode illumination component 50 properly.

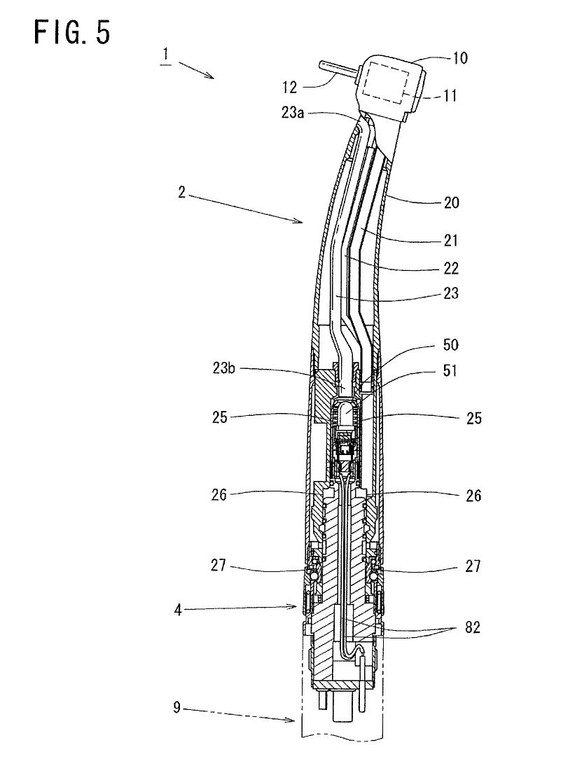

By connecting the above-described items, the dental handpiece device 1 is completed as shown in FIG. 5, which is a partially cross-sectioned side view.

Namely, the light emitting diode illumination component 50 is attached to the socket 70 at the tip of the connection component main body 80 shown in FIG. 2, and is covered with the cover 40. Thus, the connection component 4 is completed. Then, as shown in FIG. 1, the tip-side connection section 4a of the connection component 4 is inserted into the attachment hole 28 of the handpiece main body 2, and the connection ring 27 is inserted into the attachment space 84. Thus, the ball 86 contacts the outer concaved surface of the connection ring 27. In this manner, the handpiece main body 2 and the connection component 4 are rotatably attached to each other.

The rear-side connection section 4e of the connection component 4 is connected to the tube 9, and a driving medium is supplied from a medical care device (not shown) via the tube 9. Thus, the dental handpiece device 1 is driven. As the driving medium, air, water, electric power or the like is supplied. Owing to such a driving medium, the driving body 11 of the head section 10 is driven, and medical care using the dental care tool 12 is performed.

The electric power supplied from the tube 9 is supplied to the light emitting diode illumination component 50 via the electric cords 82 of the connection component 4, and thus the light emitting diode section 51 emits light. The light from the light emitting diode section 51 is incident on the waveguide body 23 from the base-side end 23b located to face the tip of the light emitting diode section 51, and is directed from the tip-side end 23a via the waveguide body 23. This light illuminates an area in the vicinity of the tip of the dental care tool 12, and the operator can perform the medical care properly.

Owing to the above-described structure, even the dental handpiece device 1 attached to a dental unit usable at any of various voltage levels can prevent the luminosity from varying.

The electric current to be supplied to the light emitting diode section 51 can be maintained at a certain level by the constant electric current diodes 57 and 58. Whichever level of voltage is applied among various voltage levels, the color tone and the luminosity of the light from the light emitting diode section 51 can be maintained at a certain level.

The diode 64 can protect the light emitting diode section 51 against a reverse voltage. Even when the light emitting diode section 51 is connected to the socket 70 in the wrong direction in terms of the positive and negative poles, the light emitting diode section 51 can be prevented from being broken.

The light emitting diode illumination component 50 can be used as being attached to the socket 70 in a conventional dental handpiece device 1.

Namely, the light emitting diode illumination component 50 can protect the light emitting diode section 51 against various environments; for example, where the light emitting diode section 51 is usable at any of various voltage levels or is connected in the wrong direction in terms of the positive and negative poles. Therefore, the light emitting diode illumination component 50 according to the present invention can be compatibly attached even to a conventional dental handpiece device 1 which generally uses a halogen lamp as a light emitting device.

When a halogen lamp is used, it does not matter in whichever direction the lamp may be connected in terms of the positive and negative poles. Therefore, the halogen lamp is usually connectable in either way. The light emitting diode illumination component 50, which uses the light emitting diode section 51, also causes no problem even if being connected in the wrong direction, owing to the above-described structure. The light emitting diode section 51 does not emit light but is not broken either, and can be re-attached in the correct direction and used with no problem.

Even if an appropriate amount of electric current is not supplied, the constant electric current diodes 57 and 58 can adjust the amount to be appropriate. Therefore, the color tone or the luminosity of the light from the light emitting diode section 51 does not reach an unexpected level, and the light emitting diode section 51 can be used properly.

The light emitting diode illumination component 50 can be structured to be compact, and so can be mounted in a small space in the dental handpiece device 1 in which the halogen lamp is conventionally located.

The dental handpiece device 1 can be used either with the light emitting diode illumination component 50 or a halogen lamp attached to the socket 70.

### EXAMPLE 2

In Example 2, a case in which an electric power control section for the light emitting diode element is provided in a connection component instead of the light emitting diode illumination component will be described.

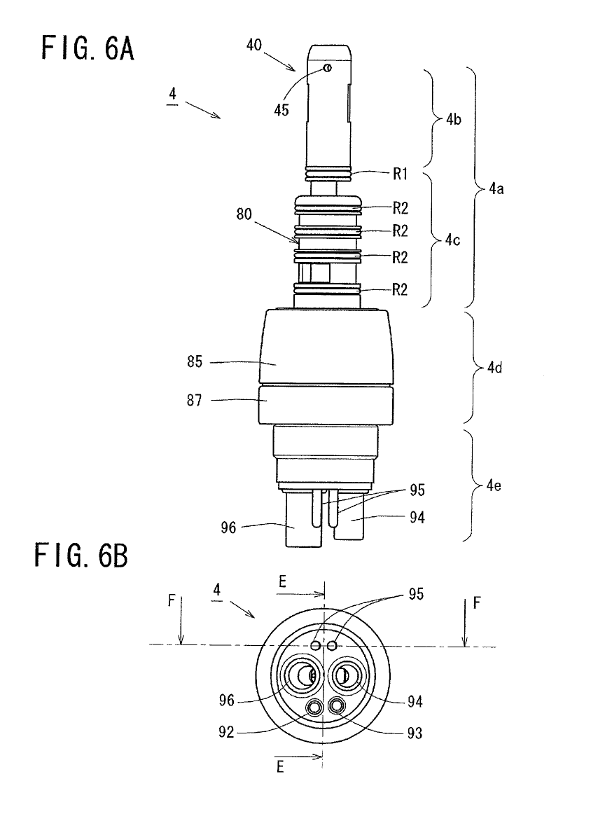

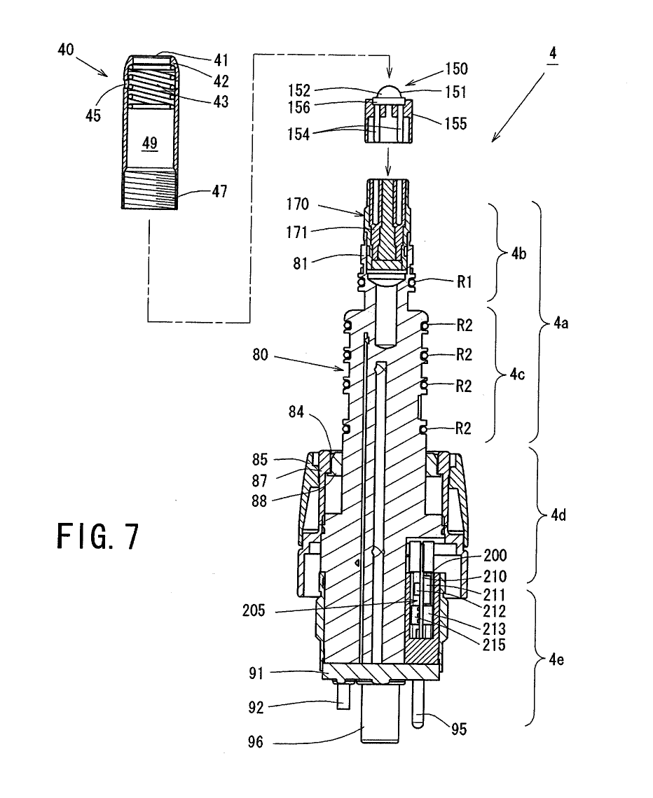

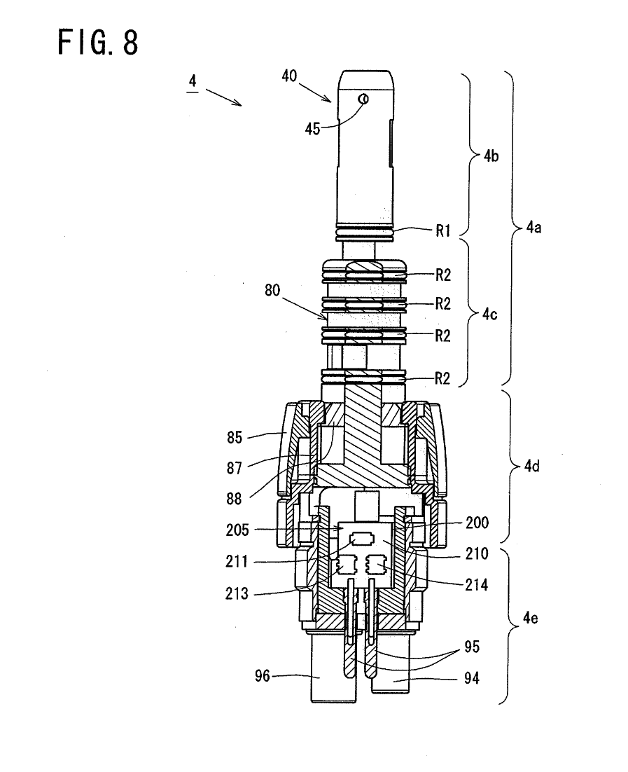

FIG. 6A is a front view of a connection component 4 in Example 2, and FIG. 6B is a bottom view thereof. FIG. 7 is a partially exploded cross-sectional view of the connection component 4 in Example 2 taken along line E-E in FIG. 6B. FIG. 8 is a cross-sectional view of the connection component 4 in Example 2 taken along line F-F in FIG. 6B.

The connection component 4 in Example 2 is the same in the basic structure and configuration as the connection component 4 in Example 1 except for detailed arrangements. In Example 2, identical elements will bear identical reference numerals to those in Example 1, and detailed descriptions thereof will be omitted. Although not shown regarding Example 2 because of the positions of the cross-sections, the ball holding ring 87 holds the ball 86 (see Example 1) and the contact cushioning ring 88 is urged by the urging means 88a (see Example 1).

The connection component 4 in Example 2 is different from that in Example 1 in the following points. As shown in FIG. 7, an electric power control section accommodating space 200 is provided in the rear-side connection section 4e of the connection component main body 80, and accommodates a substrate 210 and the like. A socket 170 which is different from the socket in Example 1 is provided in the socket attachment section 81, and a light emitting diode illumination component 150 which is different from that in Example 1 is attached to the socket 170.

The light emitting diode illumination component 150 mainly includes a light emitting diode section 151 and a cover member 155. The light emitting diode section 151 includes a light emitting diode element (not shown) on a top surface of a substrate 156 and a semispherical section 152 provided on a top surface of the light emitting diode element. From a bottom surface of the substrate 156, two connection terminals 154 are projected.

The socket 170 has two connection terminals 171 buried in a shaped resin. The connection terminals 171 are formed to have a concaved shape, and the connection terminals 154 of the light emitting diode element 151 are inserted into the concaved portions and electrically connected to the socket 170.

In the electric power control section accommodating space 200, an electric power control section 205 is accommodated. The electric power control section 205 includes a quadrangular substrate 210 and circuit elements 211 through 215 provided thereon. The circuit elements 211 through 215 are electronic devices forming a rectifier circuit, a constant electric current circuit or the like, and include a diode, a capacitor, a resistor or the like.

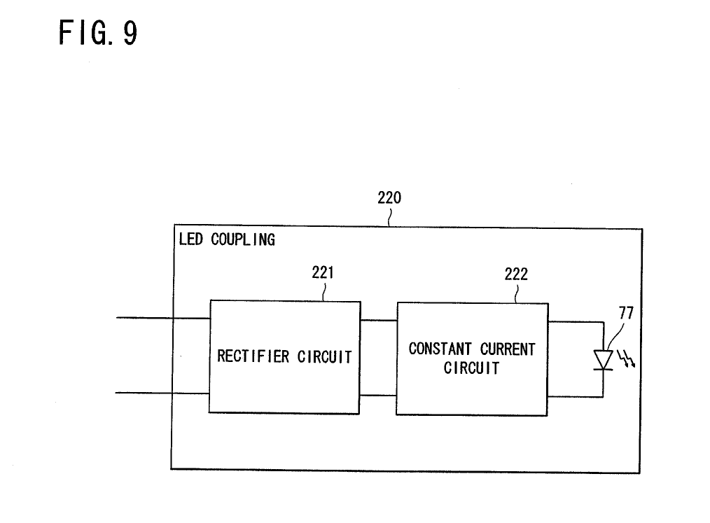

FIG. 9 is a block diagram showing a circuit configuration of an LED coupling 220 in Example 2 acting as a light emitting device.

In the LED coupling 220, a rectifier circuit 221, a constant electric current circuit 222, and a light emitting diode element 77 are connected. The rectifier circuit 221 and the constant electric current circuit 222 are provided in the electric power control section 205 (see FIG. 7), and the light emitting diode element 77 is provided in the light emitting diode illumination component 150 (see FIG. 7).

The rectifier circuit 221 rectifies a supplied AC electric current into a DC electric current and supplies the DC electric current to the light emitting diode element 77. The rectifier circuit 221 may be either a half-wave rectifier circuit or a full-wave rectifier circuit, but is preferably a full-wave rectifier circuit.

Owing to the rectifier circuit 221, in whichever direction the light emitting diode illumination component 150 may be connected to the socket 170 in terms of the positive and negative poles, the light emitting diode element 77 can be prevented from being broken by a reverse electric current and can be turned on properly. Therefore, the user can attach the light emitting diode illumination component 150 to the socket 170 for use without paying specific attention to the positive and negative poles.

The constant electric current circuit 222 stabilizes the electric current to be supplied to the light emitting diode element 77 at a certain level. Owing to this, even if the voltage level varies depending on the dental unit for which the light emitting diode illumination component 150 is used, the electric current to be supplied to the light emitting diode element 77 can be kept at a certain level, and thus the color and the luminosity of the light emitted by the light emitting diode element 77 can be kept at a certain level.

Owing to the above-described structure, the mounting space of the electric power control section 205 can be made larger than that in Example 1 in which the electric power control section is integrated in the light emitting diode illumination component 50 (see FIG. 2).

Namely, in the case where the electric power control section 205 is included in the light emitting diode illumination component 50, the electric power control section 205 is inevitably mounted in an area facing the waveguide body 23 (see FIG. 1) of the handpiece main body 2 (see FIG. 1). Therefore, the mounting space is very small.

The tip-side connection section 4a of the connection component 4 does not have an extra space almost at all because the pathways for the electric cords provided for supplying the electric power to the light emitting diode element 51 and the pathways for the driving mediums gather in this section. Therefore, mountable devices are limited, and it is difficult to mount a device of a certain size such as the rectifier circuit 221. Therefore, in Example 1, only the reverse voltage circuit 76 (see FIG. 4) is provided.

By contrast, in Example 2, the electric power control section 205 is provided at a position rearward with respect to the tip-side connection section 4a. Owing to this, a wider mounting space for the electric power control section 205 is obtained and so a relatively large device such as the rectifier circuit 221 is made mountable.

In addition, since the rectifier circuit 221 is provided, the light emitting diode illumination component 150 can be turned on regardless of the direction in which the light emitting diode illumination component 150 is attached to the socket 170 in terms of the positive and negative poles. This improves the convenience.

The light emitting diode section 151 is the only electric circuit provided in the light emitting diode illumination component 150, and the circuit elements 211 through 215 included in the electric power control section 205 are provided in the connection component 4. Therefore, a dead LED lamp can be replaced with a new one at low cost. Namely, it is not necessary to replace the circuit elements 211 through 215 and only the light emitting diode illumination component 150 accommodating the light emitting diode section 151 needs to be replaced. Therefore, the replacement can be done easily and at low cost.

The elements of the present invention and the elements in the above-described embodiment correspond as follows.

The position rearward with respect to the tip-side connection section of the present invention corresponds to the middle section 4d and the rear-side connection section 4e of the connection component 4 in the embodiment;

the tip head section corresponds to the head section 10;

the rear end of the waveguide body corresponds to the base-side end 23b;

the light emitting device corresponds to the light emitting diode illumination component 50 and the LED coupling 220;

the light emitting section corresponds to the light emitting diodes 51 and 151;

the electrode corresponds to the connection terminals 54;

the at least a part of the electric power control section corresponds to the constant electric current diodes 57 and 58;

the socket connection section corresponds to the insertion section 60;

the electric current restriction element corresponds to the constant electric current circuits 75 and 222;

the reverse voltage prevention element corresponds to the reverse voltage circuit 76 and the rectifier circuit 221; and

the driving medium corresponds to water, air, and electric power.

The present invention is not limited to the structure of the above embodiment and may be carried out in various other embodiments.

### INDUSTRIAL APPLICABILITY

The present invention is usable for a handpiece used for dental care and also for various other handpieces.

### DESCRIPTION OF THE REFERENCE NUMERALS

1 . . . dental handpiece device; 2 . . . handpiece main body; 4 . . . connection component; 4a . . . tip-side connection section; 4e . . . rear-side connection section; 4d . . . middle section; 9 . . . tube; 10 . . . head section; 12 . . . dental care tool; 23 . . . waveguide body; 23b . . . base-side end; 50 . . . light emitting diode illumination component; 51, 151 . . . light emitting diode section; 54 . . . connection terminal; 56, 205 . . . electric power control section; 57, 58 . . . constant electric current diode; 59 . . . substrate; 60 . . . insertion section; 70 . . . socket; 75, 222 . . . constant electric current circuit; 76 . . . reverse voltage circuit; 77 . . . light emitting diode element; 98 . . . driving medium pathway; 99 . . . electric cord pathway; 170 . . . socket; 220 . . . LED coupling; 221 . . . rectifier circuit

1. A light emitting device provided in a dental handpiece device for illuminating an area in the vicinity of a dental care tool at a tip of the dental handpiece device, the light emitting device comprising:
a light emitting section comprising a light emitting diode element and an electrode; and
an electric power control section for controlling electric power to be supplied to the light emitting diode element via the electrode,
wherein the electric power control section comprises:
an electric current restriction element for restricting an amount of electric current to be supplied to the light emitting diode element; and
a reverse voltage prevention element for preventing a reverse voltage from being applied to the light emitting diode element.

2. The light emitting device according to claim 1,
wherein the light emitting section and the electric power control section are integrated with each other, and
wherein the light emitting diode illumination component is integrated in the dental handpiece device.

3. The light emitting device according to claim 2, further comprising:
a substrate for carrying the electric power control section thereon,
wherein the light emitting diode element, at least a part of the electric power control section, the substrate, and the socket connection section are physically arranged in series in this order and fixed.

4. A connection component comprising:
a light emitting device according to claim 1;
a tip-side connection section provided at the one end for allowing a handpiece main body to be connected thereto detachably and pivotably around an axis;
a rear-side connection section provided at another end of the connection component for allowing a tube to be connected thereto;
a driving medium pathway provided inside the connection component for allowing a driving medium supplied from the tube via the rear-side connection section to pass therethrough and reach the handpiece main body; and
a socket provided at a tip of the tip-side connection section for allowing the light emitting section to be attached thereto in the state where the light emitting section faces a rear end of a waveguide body provided in the handpiece main body.

5. The connection component according to claim 4, wherein the electric power control section is provided rearward with respect to the tip-side connection section.

6. A dental handpiece device comprising:
a connection component according to claim 4; and
a handpiece main body connectable to the tip-side connection section of the connection component detachably and pivotably around an axis,
wherein the handpiece main body comprising:
a tip head section provided at a tip thereof for allowing a dental care tool to be attached thereto;
a driving medium pathway provided inside the handpiece main body for allowing a driving medium, supplied for driving a dental care tool, to pass therethrough; and
a waveguide body for guiding light from the light emitting diode element of the connection component and directing the light from an area in the vicinity of the tip head section toward an area in the vicinity of the dental care tool.

7. A dental handpiece device comprising:
a connection component according to claim 5; and
a handpiece main body connectable to the tip-side connection section of the connection component detachably and pivotably around an axis,
wherein the handpiece main body comprising:
a tip head section provided at a tip thereof for allowing a dental care tool to be attached thereto;
a driving medium pathway provided inside the handpiece main body for allowing a driving medium, supplied for driving a dental care tool, to pass therethrough; and
a waveguide body for guiding light from the light emitting diode element of the connection component and directing the light from an area in the vicinity of the tip head section toward an area in the vicinity of the dental care tool.
